(19) United States
(12) Patent Application Publication
(10) Pub. No.: US 2023/0240603 A1
(43) Pub. Date: Aug. 3, 2023

(54) SYSTEM AND METHOD FOR IMPROVING THE SIGNAL-TO-NOISE RATIO FOR REFLECTIVE-BASED SENSORS

(71) Applicant: Covidien LP, Mansfield, MA (US)

(72) Inventors: Jacob D. Dove, Lafayette, CO (US); Sarah L. Hayman, Boulder, CO (US); Krishna K. Chopra, Longmont, CO (US)

(21) Appl. No.: 18/159,211

(22) Filed: Jan. 25, 2023

Related U.S. Application Data
(60) Provisional application No. 63/304,264, filed on Jan. 28, 2022.

(51) Int. Cl. A61B 5/00; A61B 5/02

(52) U.S. Cl. CPC A61B 5/68; A61B 5/02

(57) ABSTRACT

The present disclosure provides systems and methods for improving signal-to-noise ratio (SNR) for a medical device sensor operating in reflective mode such that light from an emitter travels through tissue via reflection to a first detector at a first depth to provide a first detected signal over time and such that light from the emitter travels through tissue via reflection to a second detector at a second, greater depth to provide a second detected signal over time, with subtracting out the signal from superficial tissue that is common to the first and the second detected signals to provide an improved signal-to-noise ratio for the medical device sensor.

### CROSS-REFERENCE TO RELATED APPLICATIONS

This application claims the benefit of U.S. Provisional Application No. 63/304,264 filed Jan. 28, 2022, the disclosure of which is incorporated herein by reference in its entirety.

### FIELD

The present disclosure relates generally to medical devices, and more particularly to improving the signal-to-noise ratio in medical device measurements for reflective-based sensors, for example for measurements using pulse oximeters.

### BACKGROUND

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient uses attenuation of light to determine physiological characteristics of a patient. This is used in pulse oximetry, and the devices built based upon pulse oximetry techniques. Light attenuation is also used for regional or cerebral oximetry. Oximetry may be used to measure various blood characteristics, such as the oxygen saturation of hemoglobin in blood or tissue, the volume of individual blood pulsations supplying the tissue, and/or the rate of blood pulsations corresponding to each heartbeat of a patient. The signals can lead to further physiological measurements, such as respiration rate, glucose levels or blood pressure.

Oximetry is used in the clinical setting to noninvasively measure characteristics of the blood. For example, pulse oximetry typically is used to estimate arterial blood oxygenation. To estimate arterial blood oxygenation, two optical sources, typically light-emitting-diodes (LEDs), are used to inject light into the tissue. A photo-diode is used to capture the light after propagating through blood perfused tissue. During a cardiac cycle the amount of blood in the optical path changes which changes the amount the light that is absorbed. As more light is absorbed the photodiode produces less photocurrent. Hence, during the cardiac cycle the photocurrent from the photodiode is modulated. As blood oxygenation changes, the relative change in the modulated light at two distinct wavelengths changes. This relative change in the modulated photocurrent is processed (e.g., via signal conditioning, various algorithms) by the oximetry unit to estimate the arterial functional oxygenation (SpO₂).

Typically, the SpO₂ sensor is placed on a well perfused tissue site. One common location is the finger. The finger provides good signal-to-noise ratio (SNR), measured as a percent modulation of the optical (AC/DC). Here, SNR is defined as the AC/DC of the optical signal and is a measure of the amount of signal that is modulated by the blood during the cardiac cycle (AC) verse the constant signal (DC) which has not interacted with the blood perfused tissue. Hence, a higher SNR would indicate that more of the light has interacted with blood perfused tissue. While the finger provides a good SNR, placing a sensor on the finger can be an annoyance to the user. Hence other sites can be preferable, such as the wrist, chest, or back. However, these other sites are not as well perfused, which produces smaller AC signals, and have a relatively large volume of non-pulsatile tissue in the light path, which produces larger DC signals. This results in a lower signal-to-noise ratio (AC/DC), such that it becomes difficult to pick up a reliable signal to calculate arterial blood oxygenation.

What is needed in the art are improved techniques to improving the signal-to-noise ratio in medical device measurements for reflective-based sensors.

### SUMMARY

The techniques of this disclosure generally relate to improving the signal-to-noise ratio in medical device measurements for reflective-based sensors, for example for measurements using pulse oximeters.

Exemplary embodiments described herein provide systems and methods for improving signal-to-noise ratio (SNR) for a medical device sensor operating in reflective mode such that light from an emitter travels through tissue via reflection to a first detector at a first depth to provide a first detected signal over time and such that light from the emitter travels through tissue via reflection to a second detector at a second, greater depth to provide a second detected signal over time, with subtracting out the signal from superficial tissue that is common to the first and the second detected signals to provide an improved signal-to-noise ratio for the medical device sensor.

In exemplary embodiments, a photoplethysmography (PPG) sensor, such as a pulse oximeter, includes plural detectors, such as photodiodes, that are spaced at different distances from a light source, such as a light emitting diode (LED). In exemplary aspects, comparison of the natural log of the ratio of the detector furthest from the LED (which may be referred to as a deep or deeper detector) to the closer detector (which may be referred to as a shallow or shallower detector) provides a mechanism for removal of the signal due to the less perfused superficial tissue, thereby increasing percent modulation.

As is used herein, the term “reflected” refers to an arrangement wherein an emitter and a detector are on the same side of tissue, as opposed to “transmission”, e.g., used in digit sensors where the emitter and detector are on opposite sides.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 illustrates a perspective view of an exemplary patient monitoring system including a patient monitor and a patient monitoring sensor, in accordance with an embodiment;

FIG. 2 illustrates a schematic view of emitter and detector placings relative to patient tissue, in accordance with an embodiment;

FIG. 3 illustrates a graph of exemplary detected signal intensities over time for deep and shallow emitter-detector paths, in accordance with an embodiment;

FIG. 4A is an exemplary graph of normalized detector signals, in accordance with an embodiment;

FIG. 4B is an exemplary graph of percent modulations for the signals of FIG. 4A;

FIG. 5A is an exemplary graph of normalized detector signals, in accordance with an embodiment;

FIG. 5B is an exemplary graph of percent modulations for the signals of FIG. 5A; and

FIG. 6 is an exemplary watch configuration using an emitter and plural detectors placed on a line, in accordance with an embodiment.

### DETAILED DESCRIPTION

The present disclosure describes improving the signal-to-noise ratio (SNR) in medical device measurements for reflective-based sensors, for example for measurements using pulse oximeters. As will be described in more detail below, the present disclosure provides systems and methods for improving SNR for photoplethysmography (PPG) measurements, and in exemplary embodiments, for pulse oximetry measurements, wherein plural detectors, such as photodiodes, are spaced at different distances from a light source, such as a light emitting diode (LED).

In exemplary aspects, comparison of the natural log of the ratio of the detector furthest from the LED (which may be referred to as a deep or deeper detector) to the closer detector (which may be referred to as a shallow or shallower detector) provides a mechanism for removal of the signal due to the less perfused superficial tissue, thereby increasing percent modulation.

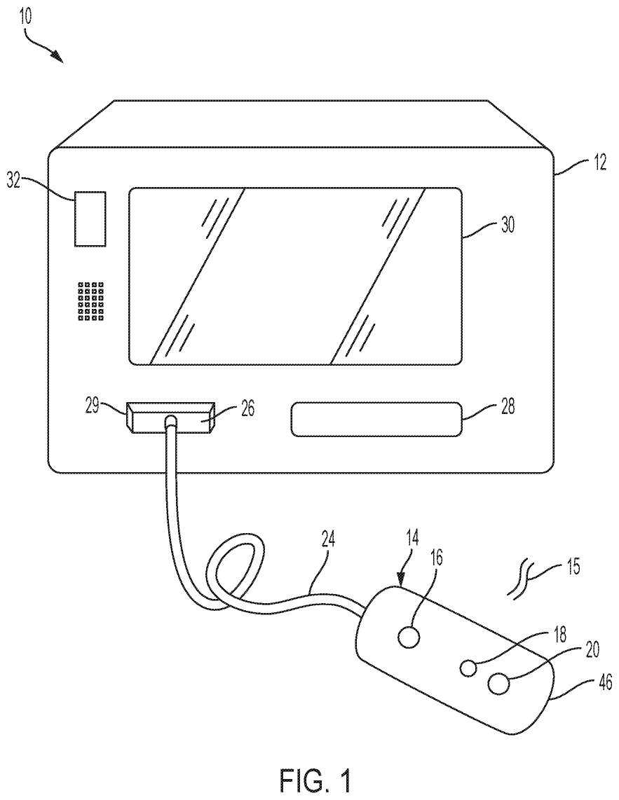

FIG. 1 illustrates an embodiment of a patient monitoring system 10 that includes a patient monitor 12 and a sensor 14, such as a pulse oximetry sensor, to monitor physiological parameters of a patient. By way of example, the sensor 14 may be a NELLCOR™, or INVOS™ sensor available from Medtronic (Boulder, Colo.), or another type of oximetry sensor. The sensors may be provided for use as a reflective-based sensor on one or more of various locations on a patient, e.g., a patient's forehead and/or temple, heel, stomach, chest, back, wrist or any other appropriate measurement site, including the fingertip, toe, or earlobe.

In the embodiment of FIG. 1, the sensor 14 is a pulse oximetry sensor that includes one or more emitters 16 configured to emit at least one wavelength of light into tissue and at least two detectors 18, 20 spaced different distances from the at least one or more emitters 16. In exemplary pulse oximetry applications, the emitter 16 transmits at least two wavelengths of light (e.g., red and/or infrared (IR)) into a tissue of the patient. In other exemplary applications, the emitter 16 may transmit 3, 4, or 5 or more wavelengths of light into the tissue of a patient. The detectors 18, 20 are photodetectors selected to receive light in the range of wavelengths emitted from the emitter 16, after the light has passed into and been reflected by at least a portion of the tissue.

The sensor 14 also includes a sensor body 46 to house or carry the components of the sensor 14. In exemplary embodiments, the body 46 includes a backing, or liner, provided around the emitter 16 and the detector 18, as well as an adhesive layer (not shown) on the patient side. The sensor 14 may be reusable (such as a durable plastic sensor), disposable (such as an adhesive sensor including a bandage/liner), or partially reusable and partially disposable.

In the embodiment shown, the sensor 14 is communicatively coupled to the patient monitor 12. In certain embodiments, the sensor 14 may include a wireless module configured to establish a wireless communication 15 with the patient monitor 12 using any suitable wireless standard. For example, the sensor 14 may include a transceiver that enables wireless signals to be transmitted to and received from an external device (e.g., the patient monitor 12, a charging device, etc.). The transceiver may establish wireless communication 15 with a transceiver of the patient monitor 12 using any suitable protocol. For example, the transceiver may be configured to transmit signals using one or more of the ZigBee standard, 802.15.4x standards WirelessHART standard, Bluetooth standard, IEEE 802.11x standards, or MiWi standard. Additionally, the transceiver may transmit a raw digitized detector signal, a processed digitized detector signal, and/or a calculated physiological parameter, as well as any data that may be stored in the sensor, such as calibration data or coefficients, such as gamma coefficients, data relating to wavelengths of the emitters 16, or data relating to input specification for the emitters 16. Additionally, or alternatively, the emitters 16 and detectors 18, 20 of the sensor 14 may be coupled to the patient monitor 12 via a cable 24 through a plug 26 (e.g., a connector having one or more conductors) coupled to a sensor port 29 of the monitor. In certain embodiments, the sensor 14 is configured to operate in both a wireless mode and a wired mode. Accordingly, in certain embodiments, the cable 24 is removably attached to the sensor 14 such that the sensor 14 can be detached from the cable to increase the patient's range of motion while wearing the sensor 14.

The patient monitor 12 is configured to calculate physiological parameters of the patient relating to the physiological signal received from the sensor 14. For example, the patient monitor 12 may include a processor configured to calculate the patient's arterial blood oxygen saturation, tissue oxygen saturation, pulse rate, respiration rate, blood pressure, blood pressure characteristic measure, autoregulation status, brain activity, and/or any other suitable physiological characteristics. Additionally, the patient monitor 12 may include a monitor display 30 configured to display information regarding the physiological parameters, information about the system (e.g., instructions for disinfecting and/or charging the sensor 14), and/or alarm indications. The patient monitor 12 may include various input components 32, such as knobs, switches, keys and keypads, buttons, etc., to provide for operation and configuration of the patient monitor 12. The patient monitor 12 may also display information related to alarms, monitor settings, and/or signal quality via one or more indicator lights and/or one or more speakers or audible indicators. The patient monitor 12 may also include an upgrade slot 28, in which additional modules can be inserted so that the patient monitor 12 can measure and display additional physiological parameters.

Because the sensor 14 may be configured to operate in a wireless mode and, in certain embodiments, may not receive power from the patient monitor 12 while operating in the wireless mode, the sensor 14 may include a battery to provide power to the components of the sensor 14 (e.g., the emitter 16 and the detector 18). In certain embodiments, the battery may be a rechargeable battery such as, for example, a lithium ion, lithium polymer, nickel-metal hydride, or nickel-cadmium battery. However, any suitable power source may be utilized, such as, one or more capacitors and/or an energy harvesting power supply (e.g., a motion generated energy harvesting device, thermoelectric generated energy harvesting device, or similar devices).

As noted above, in an embodiment, the patient monitor 12 is a pulse oximetry monitor and the sensor 14 is a pulse oximetry sensor. The sensor 14 may be placed at a site on a patient with pulsatile arterial flow. Exemplary suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and other locations described herein. The patient monitoring system 10 may include sensors 14 at multiple locations. The emitter 16 emits light which passes at least partially through the blood perfused tissue, and the detector 18 photoelectrically senses the amount of light reflected by the tissue. The patient monitoring system 10 measures the intensity of light that is received at the detector 18 as a function of time.

A signal representing light intensity versus time or a mathematical manipulation of this signal (e.g., a scaled version thereof, a logarithm taken thereof, a scaled version of a logarithm taken thereof, etc.) may be referred to as the photoplethysmography (PPG) signal. The amount of light detected may be used to calculate any of a number of physiological parameters, e.g., including oxygen saturation (the saturation of oxygen in pulsatile blood, SpO₂), an amount of a blood constituent (e.g., oxyhemoglobin), as well as a physiological rate (e.g., pulse rate or respiration rate) and when each individual pulse or breath occurs. For SpO₂, red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood, such as from empirical data that may be indexed by values of a ratio, a lookup table, and/or from curve fitting and/or other interpolative techniques.

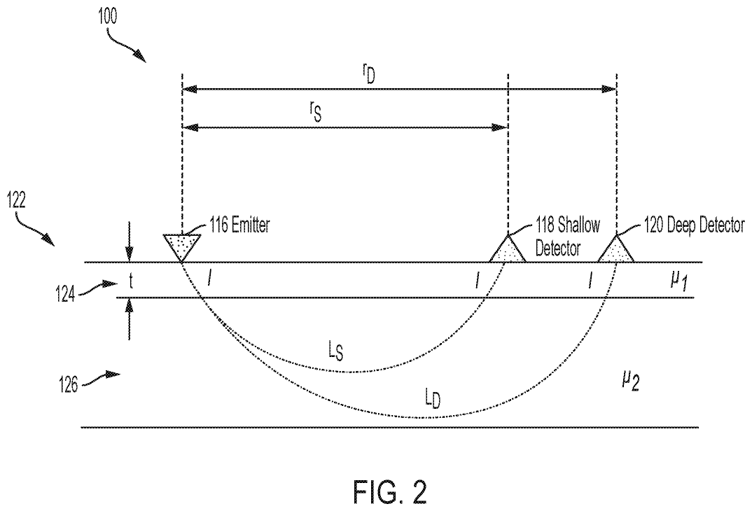

FIG. 2 illustrates an exemplary multi-detector configuration for improving SNR generally at 100 and including emitter 116 (e.g., an LED) and detectors 118, 120 (e.g., photodiodes) spaced different distances from emitter 116. In the illustrated exemplary embodiment, the three elements are provided along or approximately along the same axis (other arrangements are contemplated herein, with the two paths being somewhat or wholly out of line, though such arrangements may generate greater noise when the emitted light does not travel through the same or similar shallow tissue). In exemplary embodiments, detector 120 is furthest from the emitter 116 and may be referred to as a deep or deeper detector. Detector 118 is closer to the emitter 116 and may be referred to as a shallow or shallower detector.

Referring still to FIG. 2, shallow detector 118 is spaced a distance rₛ from the LED emitter; and deep detector 120 is spaced a distance r_{d}. from the LED emitter. The light path that is emitted from the LED and reaches the detectors has been simplified and shown as the lines, L_{S}, and L_{D}. The patient tissue, shown generally at 122, is simplified as well, with a superficial layer 124 with a thickness of t and an optical absorption coefficient of μ_{a,1} and scattering coefficient of μ_{s,1} where the extinction coefficient μ₁=μ_{a,1}+μ_{s,1}.

In exemplary embodiments, it is assumed that the superficial layer does not have any blood content. The deeper tissue 124 under the superficial layer has an absorption coefficient of μ_{a,2} and scattering coefficient of μ_{s,2}, where the extinction coefficient μ2=μ_{a,2}+μ_{s,2}, and is perfused tissue, so μ₂ changes during the cardiac cycle as the blood volume changes.

Using the Beer-Lambert law, which is a simplified method to understand the attenuation of light through the tissue as it travels from the LED to the detector, an expression for the light detected at the detector can be determined. The mean optical pathlength in tissue will depend on the emitter to detector spacing, r, and the tissue optical properties (μₐ absorption coefficient and μₛ scattering coefficient), which can be simplified to a scalar M, where M=f(r, t, μ). Using this method, the optical pathlength in the superficial tissue, l, can be expressed as M₁*t, where M₁ is the scaling factor of the superficial tissue (M₁=f(t, μ₁). Similarly, the pathlength in the perfused tissue can be expressed as Lₛ=rₛ*M₂ₛ and L_{D}=r_{D}*M_{2,d}, where M_{2,s}=f(rₛ, μ₂) and M_{2,d} f(r_{d}, μ₂). Expressions for the light on the shallow and deep detector are shown below in Equation 1 and Equation 2, respectively:

I_{S}(λ)=I₀e^{-μ}^{_{1,λ}}^{2M}^{₁}^{t-μ}^{_{2,λ}}^{M}^{₂}^{r}^{_{S}} Equation 1

I_{D}(λ)=I₀e^{-μ}^{_{1,λ}}^{2M}^{₁}^{t-μ}^{_{2,λ}}^{M}^{₂}^{r}^{_{D}} Equation 2

The natural log of the ratio of the light on the shallow detector relative to the deep detector at a wavelength of is shown in Equation 3, below:

$$R = ln ( \frac{{I}_{S} ( λ )}{{I}_{d} ( λ )} ) = ( ln ( {I}_{0} ) - {μ}_{1 , λ} 2 {M}_{1} t - {μ}_{2 , λ} {M}_{2} {r}_{S} ) - ( ln ( {I}_{0} ) - {μ}_{1 , λ} 2 {M}_{1} t - {μ}_{2 , λ} {M}_{2} {r}_{D} ) \qquad Equation 3$$

As both the deep and shallow detectors have the common signal from the superficial tissue, the difference cancels out the superficial tissue signal and Equation 3 simplifies to Equation 4, below:

R=M₂r_{D}(μ_{2,λ})−M₂r_{S}(μ_{2,λ}) Equation 4

As the blood volume changes during the cardiac cycle the absorption coefficient μ_{a,2} changes while μ_{a,1} does not. Hence, the percent modulation of the optical signal will be increased as the DC component of the signal is lowered relative to the AC.

By way of example and to examine this in more detail, the absorption coefficient of the perfused tissue, μ_{a,2}, can be represented as a simple sine function as shown in Equation 5, below:

μ_{a,2,λ}(t)=A sin(2πft)+B Equation 5

Here, A represents the change in the absorption coefficient due to the blood volume change during the cardiac cycle, f is the frequency of the heart beat (expressed here as beats per second), and B is the baseline absorption of the tissue. To demonstrate how the ratio method can increase the percent modulation of a signal, the parameters in

Table 1 were used with Equations 3-5.

TABLE 1
Exemplary parameters used to examine
percent modulation improvement.

| Parameter | Value | Units |
|---|---|---|
| A | 0.0022 | mm⁻¹ |
| B | .15 | mm⁻¹ |
| f | 1 | s⁻¹ |
| μ_{a, 2} | 0.62 | mm⁻¹ |
| M₁ | 5 | |
| M₂ | 5 | |
| r_{S} | 6 | mm |
| r_{D} | 9 | mm |
| t | 1 | mm |
| I₀ | 1 | W |

Using the parameters in

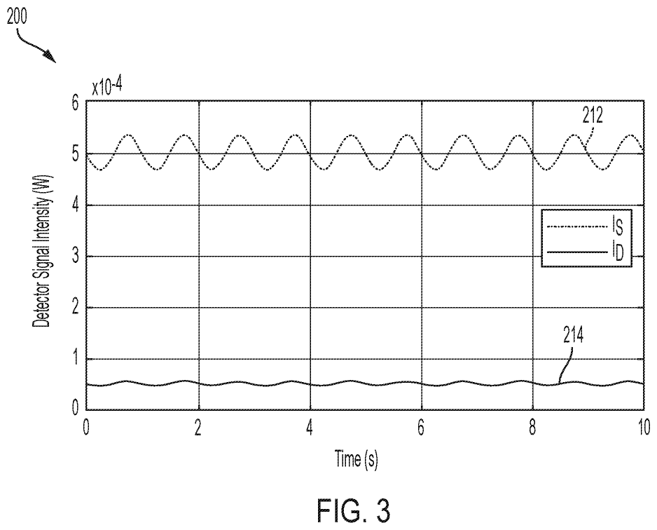

Table 1 produces signals on the shallow and deep detector as shown in FIG. 3, which plots generally at 200 detector signal intensity in watts (W) vs. time in seconds (s). Detector signals (I_{S}) for the shallow detector are shown at line 212; and detector signals (I_{D}) for the deep detector are shown at line 214. As may be seen in FIG. 3, the deep detector shows more attenuation, as the light had to propagate further to the deep detector as compared to the shallow detector.

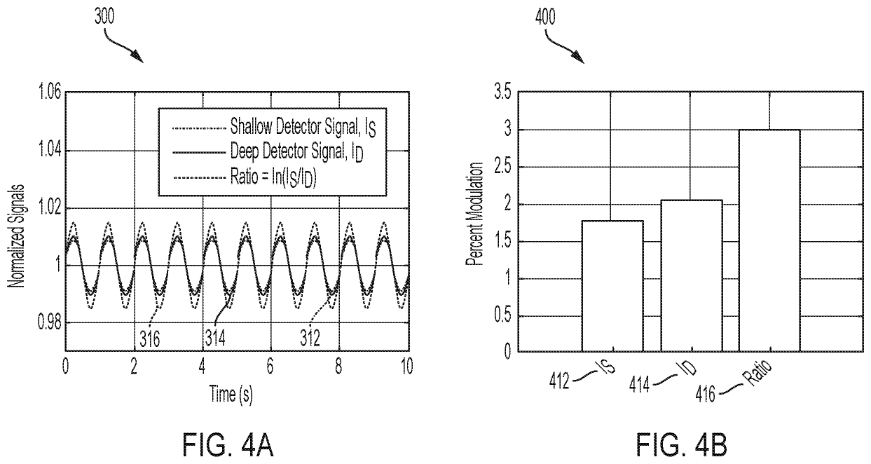

In an exemplary examination of percent modulation, the natural of logarithm of each signal may be normalized by the mean (e.g. I_{norm,s}=ln(I_{S})/mean(ln(I_{S}))), with the results shown generally at 300 in FIG. 4A, which shows normalized signals over time (the natural log of the shallow, deep, and ratio of shallow to deep detector signals) and at 400 in FIG. 4B, which shows the percent modulation of each signal. The shallow detector signal (I_{S}) is shown at line 312 in FIG. 4A. The deep detector signal (I_{D}) is shown at line 314 in FIG. 4A. The ratio of shallow to deep detector signals ln(I_{S}/I_{D}) is shown at line 316 in FIG. 4A. The percent modulation of the shallow detector signal (I_{S}) is shown at 412 in FIG. 4B. The percent modulation of the deep detector signal (I_{D}) is shown at 414 in FIG. 4B. The percent modulation of the ratio of shallow to deep detector signals ln(I_{S}/I_{D}) is shown at 416 in FIG. 4B.

In the exemplary examination shown in FIG. 4B, the ratio of the shallow to deep detector shows the largest percent modulation, followed by the deep detector; and the shallow detector had the smallest. As explained in the derivation of the ratio signal, Equations 3 and 4, the ratio signal is able to remove the non-pulsatile (or non-perfused) signal from the superficial tissue. This lowers the constant part of the signal (the DC term), while maintaining the pulsatile signal strength from the deeper tissue (AC stays the same). Hence, the percent modulation (AC/DC) is increased.

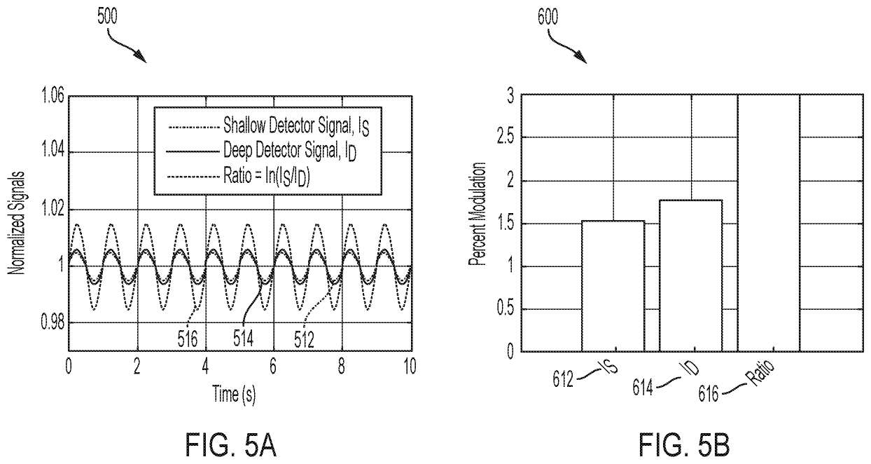

If in an exemplary embodiment, the thickness of the superficial tissue is increased to 2 millimeters (mm), the improvement SNR on the ratio signal is enhanced more compared to just the shallow or the deep detector signals, as shown generally at 500 in FIG. 5A, which shows normalized signals over time (the natural log of the shallow, deep, and ratio of shallow to deep detector signals) and at 600 in FIG. 5B, which shows the percent modulation of each signal. The shallow detector signal (I_{S}) is shown at line 512 in FIG. 5A. The deep detector signal (I_{D}) is shown at line 514 in FIG. 5A. The ratio of shallow to deep detector signals ln(I_{S}/I_{D}) is shown at line 516 in FIG. 5A. The percent modulation of the shallow detector signal (I_{S}) is shown at 612 in FIG. 5B. The percent modulation of the deep detector signal (I_{D}) is shown at 614 in FIG. 5B. The percent modulation of the ratio of shallow to deep detector signals ln(I_{S}/ID) is shown at 616 in FIG. 5B.

The effect of the superficial tissue is to increase the DC component of the signal. By increasing the thickness of the superficial tissue, the DC level on the shallow and deep detectors was increased. However, the ratio signal removes the superficial signal and is not impacted by changes.

In exemplary embodiments, the emitter-detector spacing for the shallow and deep paths may be chosen based on the anatomy of sensor location. In exemplary embodiments, an estimated depth of penetration for light propagation in the reflective configuration is about ⅓-½ the emitter-detector spacing. If the pulsatile tissue is about 3-5 mm deep (as, e.g., on the wrist), the deep emitter-detector spacing would be 6-15 mm, with the shallow being less (e.g., around 2-6 mm). For the chest or back, the pulsatile tissue may be deeper due to layers of muscle, fat, and other tissues.

Considering an exemplary wristwatch incorporating pulse oximetry measurement in accordance with aspects described herein, consideration may be made with regard to fitting 2 LEDs and detectors in a compact space. With shallow and deep signal paths, using at least 1 set of LEDs (e.g., red ˜660 nm mean wavelength (WVL) and infrared (IR) ˜900 nm mean WVL) and 2 detectors, the orientation of the detectors will be most accurate when the light travels through the same shallow tissue, so that subtraction of the shallow signal cancels.

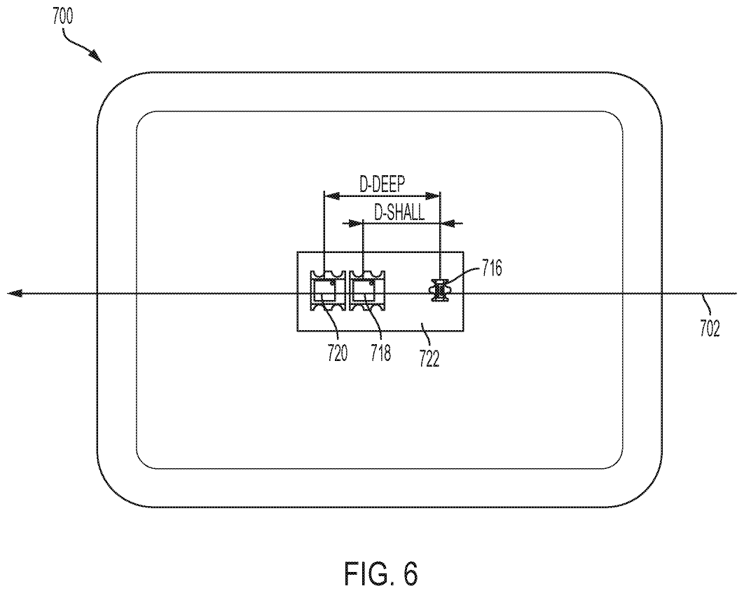

FIG. 6 shows an exemplary watch housing generally at 700, incorporating emitter 716 and detectors 718, 720 on a line (see line 702), mounted on a printed circuit board 722, with deep detector and shallow detector distances identified by D-DEEP and D-SHALL, respectively. To save space, especially with wider emitter-detector spacings, the shallow and deep detectors could be out of line and still apply the subtraction of the shallow signal, but there may be greater noise associated with such a configuration, as the shallow signal did not travel through the same tissue as the deep signal.

In exemplary aspects described herein, SNR is improved in general by providing a system and method for subtracting out the signal from a shallow signal path. This finds particularly advantageous application for challenging sites with low perfusion, such as the wrist or chest.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A method for improving signal-to-noise ratio (SNR) for a medical device sensor, comprising:
providing a medical device sensor including:
an emitter having at least one LED configured for LED emission of light through tissue;
a first detector spaced apart from the emitter at a first distance; and
a second detector spaced apart from emitter at a second, greater distance;
operating said medical device sensor in reflective mode such that light from the emitter travels through tissue via reflection to the first detector at a first depth to provide a first detected signal over time and such that light from the emitter travels through tissue via reflection to the second detector at a second, greater depth to provide a second detected signal over time; and
using a processor, subtracting out the signal from superficial tissue that is common to the first and the second detected signals to provide an improved signal-to-noise ratio for the medical device sensor.

2. A method in accordance with claim 1, wherein the sensor is a pulse oximetry sensor, and wherein the emitter emits red and infrared wavelengths.

3. A method in accordance with claim 1, wherein the processor compares the natural log of the ratio of signal from the first detector to the second detector to remove the common signal due to less perfused superficial tissue.

4. A method in accordance with claim 3, wherein the ratio is described by R=M_{b}r_{D} (μ_{2,λ})−M_{S}r_{S}(μ_{2,λ}), where the ratio is simplified because of the common signal from the superficial tissue.

5. A method in accordance with claim 1, wherein the emitter and first and second detectors are positioned within the sensor along a common axis.

6. A method in accordance with claim 1, wherein the emitter-detector spacing for each of the first and second detectors is established according to patient anatomy at a target location.

7. A method in accordance with claim 6, wherein the target location is a wrist with a pulsatile tissue depth of between about 3 and 5 millimeters deep, and wherein the spacing for the first detector is between about 2 and 6 millimeters from the emitter, and wherein the spacing for the second detector is between about 6 and 15 millimeters from the emitter.

8. A method in accordance with claim 6, wherein the emitter-detector spacing takes into account the pulsatile tissue depth at the target location and estimates a depth of penetration for light propagation in the reflective configuration of about ⅓ to ½ the emitter-detector spacing.

9. A method in accordance with claim 8, wherein the target location is the chest or the back of the patient.

10. A method in accordance with claim 7, wherein the sensor is incorporated into a watch having red and infrared LEDs and first and second detectors provided on a printed circuit board in a watch housing.

11. A system for improving signal-to-noise ratio (SNR) for a medical device sensor, comprising:
a medical device sensor including:
an emitter having at least one LED configured for LED emission of light through tissue;
a first detector spaced apart from the emitter at a first distance; and
a second detector spaced apart from emitter at a second, greater distance; and
a processor configured to:
operate said medical device sensor in reflective mode such that light from the emitter travels through tissue via reflection to the first detector at a first depth to provide a first detected signal over time and such that light from the emitter travels through tissue via reflection to the second detector at a second, greater depth to provide a second detected signal over time; and
subtract out the signal from superficial tissue that is common to the first and the second detected signals to provide an improved signal-to-noise ratio for the medical device sensor.

12. A system in accordance with claim 11, wherein the sensor is a pulse oximetry sensor, and wherein the emitter emits red and infrared wavelengths.

13. A system in accordance with claim 11, wherein the processor compares the natural log of the ratio of signal from the first detector to the second detector to remove the common signal due to less perfused superficial tissue.

14. A system in accordance with claim 13, wherein the ratio is described by R=M_{b}r_{D} (μ_{2,λ})−M_{S}r_{S}(μ_{2,λ}), where the ratio is simplified because of the common signal from the superficial tissue.

15. A system in accordance with claim 11, wherein the emitter and first and second detectors are positioned within the sensor along a common axis.

16. A system in accordance with claim 11, wherein the emitter-detector spacing for each of the first and second detectors is established according to patient anatomy at a target location.

17. A system in accordance with claim 16, wherein the target location is a wrist with a pulsatile tissue depth of between about 3 and 5 millimeters deep, and wherein the spacing for the first detector is between about 2 and 6 millimeters from the emitter, and wherein the spacing for the second detector is between about 6 and 15 millimeters from the emitter.

18. A system in accordance with claim 16, wherein the emitter-detector spacing takes into account the pulsatile tissue depth at the target location and estimates a depth of penetration for light propagation in the reflective configuration of about ⅓ to ½ the emitter-detector spacing.

19. A system in accordance with claim 18, wherein the target location is the chest or the back of the patient.

20. A system in accordance with claim 17, wherein the sensor is incorporated into a watch having red and infrared LEDs and first and second detectors provided on a printed circuit board in a watch housing.
